(19) United States
(12) Patent Application Publication
(10) Pub. No.: US 2009/0076434 A1
(43) Pub. Date: Mar. 19, 2009

(54) Method and System for Achieving Volumetric Accuracy in Hemodialysis Systems

(76) Inventors: David J. Mischelevich, Playa Del Ray, CA (US); James R. Braig, Piedmont, CA (US); Thomas P. Robinson, Encinitas, CA (US); Milan V. Treka, Northridge, CA (US); Barry N. Fulkerson, Longmont, CO (US); Cherles E. Clemens, Encinitas, CA (US); Russ T. Joseph, Las Flores, CA (US); Clark B. Foster, Mission Viejo, CA (US)

Correspondence Address: PATENTMETRIX, 14252 CULVER DR. BOX 914, IRVINE, CA, 92604, US

(21) Appl. No.: 12/210,080

(22) Filed: Sep. 12, 2008

Related U.S. Application Data
(60) Provisional application No. 60/971,937, filed on Sep. 13, 2007.

(51) Int. Cl. A61M 1/34

(52) U.S. Cl. 604/6.11

(57) ABSTRACT

Volumetric accuracy in hemodialysis systems is provided by swapping pumps between the replacement fluid side and the output side for a hemofiltration system and between the return fluid side and the sorbent side for a closed-loop, sorbent-based system, such that same quantity of fluid is pumped at each point after the end of an even number of pump swaps. A method for calculating the time interval between swaps is provided based on an allowable difference in amount pumped in the two functions at any given time. A mechanism is provided for compensating for the differences in head pressure presented to the pumps for fluid coming from the replacement-fluid containers or the reservoir and that coming back from the patient through the dialyzer. The pump-swapping system provides an accurate means that can be inexpensively implemented, including using a disposable device.

### CROSS-REFERENCE

The present application calls priority to U.S. Provisional Application No. 60/971,937 filed on Sep. 13, 2007.

### FIELD OF THE INVENTION

The present invention generally relates to dialysis systems. More specifically, the present invention relates to method for maintaining volumetric accuracy of the fluid infused into and removed from the patient during dialysis.

### BACKGROUND OF THE INVENTION

A dialysis system typically includes a hydraulic system for circulating blood, a hydraulic system for circulating dialysate fluid, and a semi-permeable membrane. Urea and other blood components, but not blood cells, travel across the membrane from the blood side to the dialysate side as the blood and dialysate fluid both flow past the membrane.

In the most common type of hemodialysis systems, dialysate fluid flows through the dialyzer in a single pass and is discarded into a drain. The dialysate fluid remains entirely within the dialysate circuit and, except possibly for some convection of fluid from the dialysate side of the dialyzer membrane to the blood side of the dialyzer membrane, no dialysate fluid is infused into the patient. In an alternative mode, called hemofiltration, I.V.-quality replacement fluid is infused into the patient by a direct connection between the dialysate circuit and the blood circuit (by-passing the dialyzer) and an equal amount of fluid is removed from the patient by taking the fluid off through the dialyzer and discarding it. In this approach, an additional amount of fluid in the form of ultrafiltrate may be optionally removed to obtain a net removal of fluid from a fluid-overloaded patient.

An alternative to single-pass hemodialysis systems uses a closed-loop dialysate circuit in which instead of the dialysate fluid being constantly discarded during the treatment, the dialysate is recycled. As dialysate fluid is recycled, urea and other blood waste compounds must be removed by passing it through a sorbent cartridge before the fluid is again passed by the membrane.

In order to ensure that hemofiltration is completely safe, the amount of replacement fluid infused into the patient must be equal to the amount of fluid being removed from the patient, referred to herein as output fluid. Thus, it is critical to maintain volumetric accuracy during the dialysis procedure. This requires accurate determination of the volume of replacement fluid being infused, as well as the volume of output fluid being removed from the patient. The same holds true for closed-loop, sorbent-based systems, where volumetric accuracy is required to be maintained between the return fluid side and the sorbent side.

Methods are provided in the prior art for obtaining volumetric accuracy of the replacement fluid and output fluid during the process of dialysis. One prior art approach for maintaining volumetric accuracy involves weighing both the replacement fluid and output fluid. However, this approach is difficult to implement in practice as it is very inconvenient for users to move the system with the bags swinging on measuring scales. Another prior art method comprises the use of volumetric balance chambers for hemodialysis systems. Such chambers are, however, complex and expensive to build and also not suitable for disposable devices. Volumetric flow measurements are another known method, but the accuracy of this method is not proven. Further, this method is very difficult to implement for a hemodialysis system in disposable form. Another prior art approach involves using two piston pumps to achieve volumetric accuracy. However, this approach is extremely difficult to implement at a reasonable cost in disposable form, and is also not economical to operate at the required pumping volumes, which are of the order of 200 ml/min.

Thus, there is no satisfactory mechanism in the prior art for continually maintaining volumetric accuracy during the dialysis process that can be easily implemented at a reasonable cost. Further, most of the prior art methods for maintaining volumetric accuracy of replacement fluid and output fluid are not suited for use with disposable devices.

There is therefore a need for a method and a system that can be used to accurately maintain the volume of the fluid infused into and removed from the patient, and which can be implemented inexpensively.

Also required is a system for maintaining volumetric accuracy, which can be employed with disposable devices as well as with standard dialysis equipment.

### SUMMARY OF THE INVENTION

In the present invention, volumetric accuracy in hemodialysis systems is achieved by swapping pumps used on the replacement fluid side and on the output side so that same quantity of fluid is pumped at each point after an even number of swaps. Because a time gap is created during a pump swap, it is necessary to calculate the time interval between swaps. This calculation is a function of the maximum allowable difference in the amount pumped, as determined by two functions, at any given time. The calculation must compensate, however, for differences in head pressure presented to the pumps for fluid coming from the replacement-fluid containers and that coming back from the patient through the dialyzer. The present invention provides an accurate means for achieving volumetric accuracy that can be inexpensively implemented in the form of a disposable device. It can be employed with hemofiltration systems as well as with closed-loop, sorbent-based systems.

In one embodiment, the pumps used are peristaltic pumps, although other pumps known to persons of ordinary skill in the art may be used. In one embodiment, two pumps are used, pumps A and B, for pumping replacement fluid and output fluid respectively. Pump A delivers more fluid per unit time than pump B. Conventionally, this would result in more replacement fluid being pumped than output fluid in any given period of time.

To achieve volumetric balance between the replacement fluid and output fluid, the pumps are swapped every T minutes. At the end of the first T minute interval, pump A has delivered more volume, Q, than pump B. Specifically, in one embodiment, the present invention has two sub-circuits in the dialysate circuit, R for replacement fluid and O for fluid coming back from the patient through the dialyzer. In the first pumping interval fluid, replacement fluid is routed through Pump A and output fluid is routed through Pump B. At the end of time interval T, Q more replacement fluid has been pumped in R than output fluid in O. When pumps A and B are swapped in the second interval and output fluid in O is pumped by Pump A and replacement fluid in R is pumped by pump B, Q less replacement fluid in R will have been pumped than output fluid in O. Therefore, at end of the second interval (and at the end of an even number of swaps), the difference will Q−Q=0, thereby achieving volumetric balance between replacement and output fluid. Because there may be some, usually small, change in the flow rate through a pump over time, and the volume delivered per unit time changes, the difference may not be exactly zero, but likely close to zero.

Since the fluid volumes delivered by a pump, such as a peristaltic pump, will vary with the head pressure, the present invention provides a means to compensate for differences in head pressure occurring in sub-circuits R and O under uncompensated conditions. A method for calculating the time interval between swaps is also provided based on the defined maximum allowable difference in amount pumped in the two functions at any given time.

In one embodiment, the present invention is directed to a system for maintaining volumetric balance of the fluid infused into a patient and the fluid removed from said patient during renal dialysis, the system comprising a first fluid circuit for infusing fluid into the patient; a second fluid circuit for removing fluid from the patient; a first pump configured to alternately operate on said first circuit and said second circuit; a second pump configured to alternately operate on said second circuit and said first circuit; and a controller for causing said first pump to alternatively operate on said first circuit and said second circuit and for causing said second pump to alternatively operate on said first circuit and said second circuit, wherein each of the said first pump and second pump operate only one circuit at a given time.

Optionally, the first pump delivers a higher amount of fluid per unit time than the second pump. The first and second pumps alternately operate on the first and second circuits for a time interval ‘T’, wherein ‘T’ is derived from an allowable difference in the amount of fluid delivered per unit time by the first and second pumps. Optionally, the first and second pumps are peristaltic pumps. Optionally, the system is implemented in the form of a disposable device. Optionally, the system further comprises an active restrictor for equalizing a pressure differential between the first and second circuits. The restrictor equalizes said pressure differential based upon a measured pressure differential derived from a first pressure sensor in said first circuit and from a second pressure sensor in said second first circuit.

In another embodiment, the present invention is a method for maintaining volumetric balance of the fluid infused into a patient and the fluid removed from the patient during renal dialysis, the method comprising using a first fluid circuit for infusing fluid into the patient; using a second fluid circuit for removing fluid from the patient; providing a first pump having an ability to alternately operate on the first circuit and the second circuit; providing a second pump having an ability to alternately operate on the second circuit and the first circuit; causing the first pump to alternatively operate on the first circuit and the second circuit; and causing the second pump to alternatively operate on the first circuit and the second circuit, wherein each of the first pump and second pump operate only one circuit at a given time. The first pump delivers a higher amount of fluid per unit time than the second pump. The first and second pumps alternately operate on the first and second circuits for a time interval ‘T’, wherein ‘T’ is derived from an allowable difference in the amount of fluid delivered per unit time by the first and second pumps. The method is implemented by a disposable device. Optionally, the method further comprises the step of equalizing a pressure differential between the first and second circuits. The pressure differential is determined based upon a measured pressure differential derived from a first pressure sensor in the first circuit and from a second pressure sensor in the second first circuit.

### BRIEF DESCRIPTION OF THE DRAWINGS

These and other objects and advantages of the present invention will become more fully apparent from the following detailed description when read in conjunction with the accompanying drawing with like reference numerals indicating corresponding parts, wherein:

FIG. 1 provides a schematic diagram of one embodiment of the present invention as applied to hemofiltration;

FIG. 2 provides a schematic diagram of another embodiment of the present invention, as applied to closed-circuit sorbent-based hemodialysis; and

FIG. 3 is a table illustrating the pump swapping intervals for different pumping rates, to achieve a given volumetric accuracy in the system of present invention.

### DETAILED DESCRIPTION OF THE INVENTION

The present invention is directed towards novel methods and systems for maintaining volumetric accuracy of replacement fluid and output fluid in a hemodialysis system. In one embodiment, the method of present invention involves swapping pumps used at the replacement fluid side and on the output side such that an equal quantity of fluid is pumped at each side. The pump-swapping system of the present invention provides an accurate means for maintaining the fluid volumes during the dialysis procedure, and can be inexpensively implemented, for reusable as well as disposable devices.

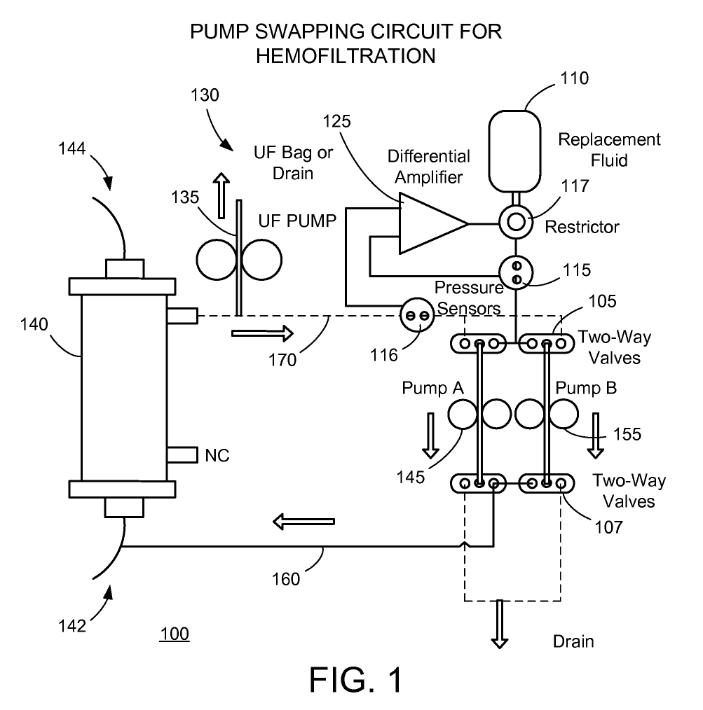

FIG. 1 illustrates an exemplary pump swapping circuit as employed in the system of the present invention. Referring to FIG. 1, a pump swapping circuit 100 for hemofiltration comprises two pumps, Pump A 145 and Pump B 155. These two pumps are in fluid communication with the replacement fluid circuit R 160 and the output fluid circuit O 170. The fluid communication is facilitated by means of two pairs of two-way valves 105 and 107. For the replacement fluid circuit R 160, a replacement fluid source 110 provides fluid through a restrictor 117 to the pair of two-way valves 105. Thereafter, depending on which of the two valves in the pair 105 is open, the replacement fluid is pumped by either Pump A 145 or Pump B 155 to the second set of two-way valves 107. This set of two-way valves 107 channelizes the replacement fluid to the replacement circuit R 160, which is in fluid communication with the output 142 of the dialyzer 140. In the present embodiment, the communication with the output 142 of the dialyzer 140 is a post-dialyzer infusion configuration. In another configuration known in the art, the communication is with the input 144 of the dialyzer instead. One of ordinary skill in the art would appreciate that either configuration may be used, without impacting the scope of the invention.

The pair of two-way valves 105 can be configured to alternatively open such that any of the following fluid communication paths may be established:
Between output fluid circuit O 170 and Pump A 145;
Between replacement fluid circuit R 160 and Pump B 155;
Between replacement fluid circuit R 160 and Pump A 145; and
Between output fluid circuit O 170 and Pump B 155.

The system 100 also comprises two pressure sensors 115 and 116. The sensor 116 is located on the output circuit O 170 while the sensor 115 is located proximate to the replacement fluid source 110. The pressure sensors 115 and 116 are used for monitoring pressure. The pressure data from these sensors is provided to the active restrictor 117, via a differential amplifier 125. Depending on the pressure measurements, the restrictor 117 variably restricts the flow of replacement fluid as required.

During dialysis, additional fluid may be removed from the patient if required, in the form of ultrafiltrate (UF). For this purpose, a UF pump 135 is provided, that pumps the UF to a bag or drain 130. Since UF fluid is removed prior to the point of pressure measurement in the output fluid sub-circuit O 170, volumetric accuracy is maintained irrespective of how much or how little UF is removed.

Operationally, volumetric accuracy in the hemodialysis system of the present invention is achieved by swapping the pumps 145 and 155 used on the replacement fluid side and on the output side so that same quantity of fluid is pumped at each point after an even number of swaps. The two pairs of two-way valves 105 and 107 facilitate the use of each of the pumps alternatively with the replacement fluid circuit R 160 and the output fluid circuit O 170.

In one embodiment, the pumps used are peristaltic pumps. One of ordinary skill in the art would appreciate that other types of pumps may also be used, since volumetric balance in renal dialysis is achieved by the use of pump-swapping technique, and is not dependent on the type of pump. In one embodiment, Pump A 145 delivers more fluid per unit time than pump B 155. Therefore, this would result in more replacement fluid being pumped than output fluid in any given period of time.

One of ordinary skill in the art would appreciate that pumps that include a disposable element can have a pumping rate differential since volumes across disposable elements are not equal, even if they are of the same size and type. For example, the volumes of two disposable syringes of nominally the same size inserted within two syringe-pump assemblies will not be exactly the same. One of ordinary skill in the art would also appreciate that two pumps that do not have disposable elements can usually be tuned so there will be no differential in pumping rate between the two.

Examples of pumps using disposable elements that can be implemented with the present invention include, but are not limited to, rotary or linear peristaltic pumps, syringe pumps, rotary vane pumps, centrifugal pumps, and diaphragm pumps.

To achieve volumetric balance between the replacement fluid and output fluid, the pumps 145 and 155 are swapped every T minutes. At the end of the first ‘T’ minute interval, owing to the pump's specific characteristics, pump A 145 would deliver more volume than pump B 155. The fluid volume delivered by pump A 145 is termed as ‘Q’. Thus, if during the first pumping interval ‘T’, replacement fluid is routed through Pump A 145 and output fluid is routed through Pump B 155, then at the end of time interval T, Q more replacement fluid would have been pumped in the replacement fluid circuit R 160 than output fluid in the circuit O 170. Thereafter, pumps A 145 and B 155 are swapped in the next time interval and output fluid in circuit O 170 is pumped by Pump A 145 and replacement fluid in circuit R 160 is pumped by pump B 155. In this interval, ‘Q’ less replacement fluid in R 160 will be pumped than output fluid in O 170. Therefore, at end of the second interval (and at the end of an even number of swaps), the difference in volume pumped during each interval would be: Q−Q=0. Thus the net volume difference is zero after an even number of swaps, thereby achieving volumetric balance between the replacement fluid infused and the output fluid coming back from the patient through the dialyzer. One of ordinary skill in the art would appreciate that there may be a minute change in the flow rate through a pump over time, and consequently, in the volume delivered per unit time. In that case, the net volume difference may not be exactly zero, but very close to zero.

### Compensation for Head Pressure Differences

The volume pumped by a peristaltic pump depends on head pressure. Head pressure for the pumps is a function of the sub-circuit, not the pump, and is systematically different in the replacement fluid circuit R 160 versus the output circuit O 170. It is thus necessary to equalize head pressures experienced by Pump A 145 and Pump B 155.

In one embodiment, head pressures are equalized by modulating the restrictor 117 on the input circuit from the replacement fluid source 110. The restrictor modulation is achieved based on the output of a differential amplifier 125, which calculates pressure differentials between the pressure values measured by head pressure sensors 115 and 116 located between the pumps 145 and 155. The amount of compensation required will depend on how much a pump is influenced by head pressures in the replacement fluid circuit R 160 and the output fluid circuit O 170. The head pressure in circuit O 170 will typically be negative. The head pressure in circuit R 160 will be positive if the replacement fluid bags (source) 110 are elevated above the level of the pumps and negative if the bags are vertically positioned below the level of the pumps. For pumps utilizing heavy duty pump tube segments, the differences may be relatively small.

As mentioned, head pressures are equalized by measuring the pressures in the sub-circuits R 160 and O 170, providing those pressures as input to a differential amplifier 125, and modulating the inflow from the replacement fluid bag 110 with a variable restrictor 117 in sub-circuit R 160 that is regulated by the output of the differential amplifier 125. Since the head pressure is a function of the sub-circuit rather than the pump, therefore, it is necessary to regulate the average difference between the head pressures of the two sub-circuits in an unregulated state. The pressures in the unregulated state can be measured initially and at desired intervals during operation by briefly turning off regulation. This recalibration does not require stopping pumping.

In one embodiment, pump head pressures can vary from zero to over several hundred mmHg, depending on the dialyzer incorporated, the height of the replacement fluid relative to the dialysis machine and the dialysate flow rate setting. For example, for a dialysate flow of 200 ml/min. and replacement fluid bags hung 5-10 inches above the dialysis machine, the pressure differentials are in the range of 10 mmHg. In general, when the pressure in replacement circuit R 160 is higher than the pressure in circuit O 170, the flow restrictor 117 will restrict flow from the replacement fluid source 110 in order to compensate for the pressure differential.

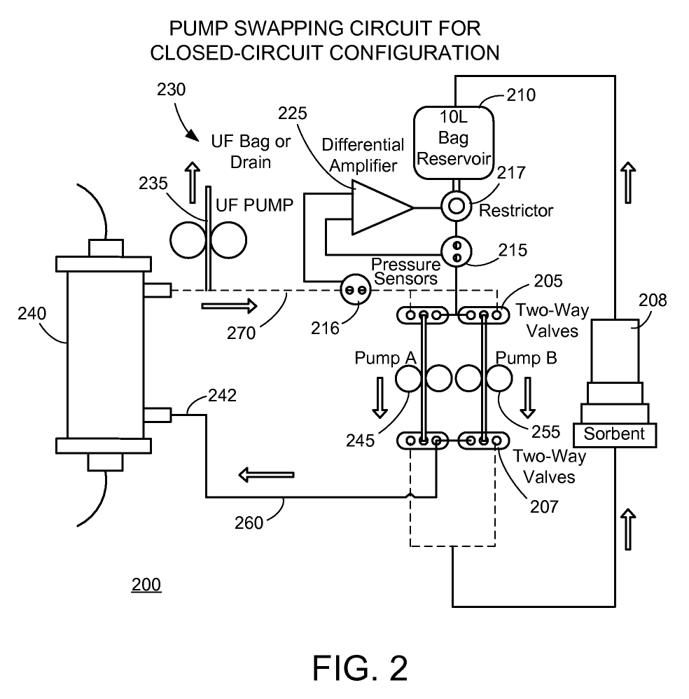

In another embodiment, the present invention is applied to a hemodialysis system that uses a closed-loop dialysate circuit. The dialysis system in this embodiment is sorbent based, in which instead of the dialysate fluid being constantly discarded during the treatment, it is recycled by passing through a sorbent cartridge for removal of substances such as urea. FIG. 2 illustrates this embodiment of the present invention with an alternative pump swapping circuit.

Referring to FIG. 2, the pump swapping circuit 200 for hemofiltration comprises two pumps, Pump A 245 and Pump B 255. These two pumps are in fluid communication with the return fluid circuit R 260 and the sorbent fluid circuit S 270. The fluid communication is facilitated by means of two pairs of two-way valves 205 and 207. For the return fluid circuit R 260, a reservoir fluid source 210 provides fluid through a restrictor 217 to the pair of two-way valves 205. Thereafter, depending on which of the two valves in the pair 205 is open, the replacement fluid is pumped by either Pump A 245 or Pump B 255 to the second set of two-way valves 207. This set of two-way valves 207 channelizes fluid through a sorbent cartridge 208 and through the reservoir 210 to the return circuit R 260, which is in fluid communication with the input port 242 of the dialyzer 240.

The pair of two-way valves 205 can be configured to alternatively open such that any of the following fluid communication paths may be established:
Between sorbent fluid circuit S 270 and Pump A 245;
Between return fluid circuit R 260 and Pump B 255;
Between return fluid circuit R 260 and Pump A 245; and
Between sorbent fluid circuit S 270 and Pump B 255.

The system 200 also comprises two pressure sensors 215 and 216. The sensor 216 is located on the sorbent circuit S 270 while the sensor 215 is located proximate to the reservoir fluid source 210. The pressure sensors 215 and 216 are used for monitoring pressure. Pressure data from these sensors is provided to the active restrictor 217, via a differential amplifier 225. Depending on the pressure measurements, the restrictor 217 variably restricts the flow of reservoir fluid as required.

As in previous embodiment, this embodiment too has a provision of a UF (ultrafiltrate) pump 235, so that additional fluid in the form of (UF) may be removed from the patient during dialysis, if required. The UF pump 235 pumps the ultrafiltrate to a bag or drain 230. Since UF fluid is removed prior to the point of pressure measurement in the sorbent fluid sub-circuit S 270, volumetric accuracy is maintained irrespective of how much or how little UF is removed.

Operationally, volumetric accuracy in the hemodialysis system of the present invention is achieved by swapping the pumps 245 and 255 used on the return fluid side and on the sorbent side so that same quantity of fluid is pumped at each point after an even number of swaps. The two pairs of two-way valves 205 and 207 facilitate the use of each of the pumps alternatively with the return fluid circuit R 260 and the sorbent fluid circuit S 270.

In one embodiment, the pumps used are peristaltic pumps. One of ordinary skill in the art would appreciate that other types of pumps may also be used, since volumetric balance in renal dialysis is achieved by the use of pump-swapping technique, and is not dependent on the type of pump. In one embodiment, Pump A 245 delivers more fluid per unit time than pump B 255. Therefore, this would result in more return fluid being pumped than sorbent fluid in any given period of time.

One of ordinary skill in the art would appreciate that pumps that include a disposable element can have a pumping rate differential since volumes across disposable elements are not equal, even if they are of the same size and type. One of ordinary skill in the art would also appreciate that two pumps that do not have disposable elements can usually be tuned so there will be no differential in pumping rate between the two.

To achieve volumetric balance between the return fluid and sorbent fluid, the pumps 245 and 255 are swapped every T minutes. At the end of the first ‘T’ minute interval, owing to the pump's specific characteristics, pump A 245 would deliver more volume than pump B 255. The fluid volume delivered by pump A 245 is termed as ‘Q’. Thus, if during the first pumping interval ‘T’, reservoir fluid is routed through Pump A 245 and sorbent fluid is routed through Pump B 255, then at the end of time interval T, Q more reservoir fluid would have been pumped in the return fluid circuit R 260 than sorbent fluid in the circuit S 270. Thereafter, pumps A 245 and B 255 are swapped in the next time interval and sorbent fluid in circuit S 170 is pumped by Pump A 245 and return fluid in circuit R 260 is pumped by pump B 255. In this interval, ‘Q’ less reservoir fluid in R 260 will be pumped than sorbent fluid in S 270. Therefore, at end of the second interval (and at the end of an even number of swaps), the difference in volume pumped during each interval would be: Q−Q=0. Thus the net volume difference is zero after an even number of swaps, thereby achieving volumetric balance between the return fluid infused and the sorbent fluid coming back from the patient through the dialyzer. Again, since there may be some, usually small, change in the flow rate through a pump over time, so that the volume delivered per unit time changes, the net volume difference may not be exactly zero at times, but substantially close to zero.

### Compensation for Head Pressure Differences

As is true for the embodiment shown in FIG. 1, the volume pumped by a peristaltic pump in the embodiment illustrated in FIG. 2 depends on head pressure. Further, since head pressure for the pumps is a function of the sub-circuit, not the pump, and is systematically different in the return fluid circuit R 260 versus the sorbent circuit S 270, therefore it is necessary to equalize head pressures experienced by Pump A 245 and Pump B 255.

In one embodiment, head pressures are equalized by modulating the restrictor 217 on the input circuit from the reservoir fluid source 210. The restrictor modulation is achieved in a similar manner as with the embodiment of FIG. 1, and is based on the output of a differential amplifier 225. The differential amplifier 225 calculates pressure differentials between the pressure values measured by head pressure sensors 215 and 216 located between the pumps 245 and 255. The amount of compensation required will depend on how much a pump is influenced by head pressures in the return fluid circuit R 260 and the sorbent fluid circuit S 270. The head pressure in circuit S 270 will typically be negative. The head pressure in circuit R 260 will be positive if the reservoir 210 is elevated above the level of the pumps and negative if the reservoir is vertically positioned below the level of the pumps. For pumps utilizing heavy duty pump tube segments, the differences may be relatively small.

As mentioned, head pressures are equalized by measuring the pressures in the sub-circuits R 260 and S 270, providing those pressures as input to a differential amplifier 225, and modulating the inflow from the reservoir 210 with a variable restrictor 217 in sub-circuit R 260 that is regulated by the output of the differential amplifier 225. Since the head pressure is a function of the sub-circuit rather than the pump, therefore, it is necessary to regulate the average difference between the head pressures of the two sub-circuits in an unregulated state. The pressures in the unregulated state can be measured initially and at desired intervals during operation by briefly turning off regulation. This recalibration does not require stopping pumping.

In one embodiment pump head pressures may vary from zero to over several hundred mmHg, depending on the dialyzer incorporated, the height of the reservoir relative to the dialysis machine and the dialysate flow rate setting. For example, pressure differentials are in the range of 10 mmHg for a dialysate flow of 200 ml/min. and with the reservoir located 5-10 inches above the pumps of the dialysis machine. When pressure in circuit R (return) 260 is higher than pressure in circuit S 270 (from dialyzer), the flow restrictor 217 restricts flow from the reservoir 210 to compensate.

In either the configuration in FIG. 1 or the one in FIG. 2, at times there may be increased outflow into the dialysate circuit segment (O 170 or S 270 respectively), due to increased dialyzer trans-membrane pressure (TMP). This may happen, for example, because of an outflow obstruction of dialyzer (140 or 240 respectively). In such a case, there may be the possibility of the restrictor (117 or 217 respectively) not being able to open up sufficiently to regulate, for example if the replacement fluid source 110 or reservoir 210 is located below the level of the pumps. To counter this, a booster pump may be inserted in the circuit after the replacement fluid source 110 or the reservoir 210. The booster pump may be configured to be turned on automatically in case the differential amplifier (125 or 225, respectively) and/or the restrictor (117 or 217, respectively) is unable to regulate the system.

### Determination of Pump-Swapping Interval

Since a time gap is created during a pump swap, therefore it is necessary to calculate the time interval between swaps. This calculation is a function of the maximum allowable difference in the amount of fluid pumped, as determined by two functions, at any given time. The calculation must compensate, however, for differences in head pressure presented to the pumps for fluid coming from the replacement-fluid containers and that coming back from the patient through the dialyzer.

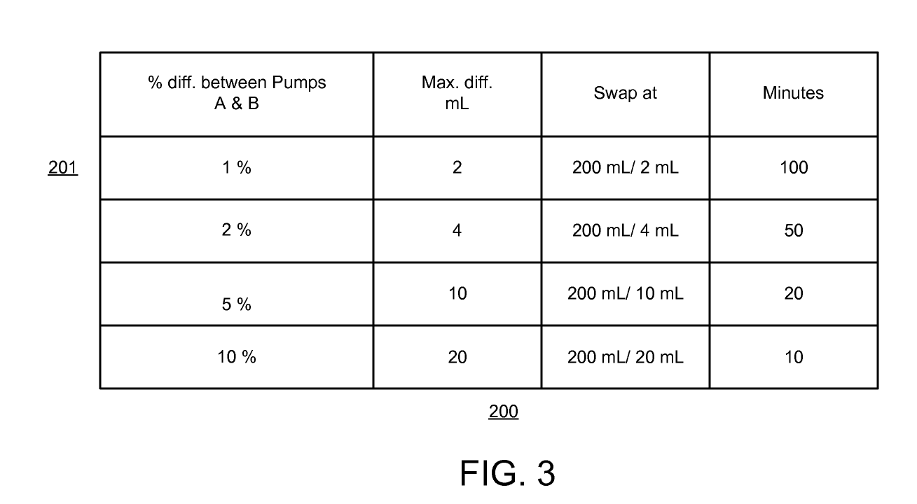

The frequency at which the pumps are swapped depends on the maximum acceptable increase or decrease in fluid volume in a patient during the dialysis process for any given interval T. For example, if the allowable net gain or loss is 200 ml and the replacement fluid is being input at a rate of 200 ml/min, then the pump swapping frequency for various levels of differences in the pumping rate of the two pumps are detailed in a table 300 in FIG. 3.

The following description refers to the components in the embodiment shown in FIG. 1, but is also applicable in the same manner to the embodiment illustrated in FIG. 2. Referring to FIG. 3, the first row 301 of the table illustrates that when the percentage difference in the pumping rates of the two pumps—pump A 145 and pump B 155 is 1%, which amounts to a fluid volume difference of 2 ml (for an allowable net gain or loss of 200 ml), then swapping the pumps at a time interval of 200 ml/2 ml=100 minutes would achieve zero volumetric difference. Similarly, for a pumping rate difference of 2%, swapping the pumps at an interval of 200 ml/4 ml=50 minutes would achieve volumetric balance, and so on. This is illustrated in the subsequent rows of table 300.

Even if a much more stringent limit was to be put on the maximum volume of fluid that can be infused into or removed from a patient—such as ±30 ml as opposed to ±200 ml in the above example, the swap interval for the case when the pumping difference is 5%, would be 30 ml/10 ml=3 minutes. Since only switching the two-way valves (shown as 105 in FIG. 1) is needed for swapping the pumps and starting and stopping the pumps is not required, even a short interval of 3 minutes (or a shorter) is practically implementable.

Swapping the pumps more frequently can also mitigate any divergence in pump tube performance. Since in the system of present invention, the tubes both the pumps are subject to the same number of impacts, therefore the performance of the pumps tends not to diverge.

When using the pump-swapping approach, if the process does not stop at an even number of swaps it could result in a differential error in the volumetric balance of the replacement fluid and the output fluid. Therefore, in one embodiment, the system is configured to stop only when an even number of swaps are completed, unless the system is overridden. The potential impact of the problem ending in a net differential error can also be reduced by swapping the pumps more frequently. In any case, it can be guaranteed that any net difference will not be outside the originally set boundary for maximum allowable net fluid loss or gain, such as ±200 ml. Therefore, in one embodiment, the present invention comprises a controller in data communication with all operative pumps. The controller comprises software with a counter that tracks, by increment, the number of pump swaps. Where the number of pump swaps is uneven, the controller implements a blocking signal which prevents the system from being shut down. The controller releases the blocking signal when the counter is an even number, thereby permitting a shutdown of the system. The controller is further responsible for transmitting the swapping signal which causes the appropriate valves to open and close, thereby effectuating the pump swap.

### Residual

During the process of pump swapping, there will be a small amount of residual fluid that will shift from one sub-circuit to the other. For example, if the peristaltic pump tubing is 0.8 ml/inch and the pump-tube segment length is 3 inches, the residual would be 2.4 ml (3 inch×0.8 ml/in=2.4 ml) per each time period. In an exemplary time period of 50 minutes, and with a pumping rate of 200 ml/min, 10 liters of fluid (50 min×200 ml/min=10,000 ml) will be pumped. Therefore, the percentage of residual to the total fluid pumped in liters is only 0.024% (2.4 ml/10,000 ml=0.024%). The effect of even this small percentage of residual will be nullified, because a shift between the sub-circuits occurs due to pump swapping, which cancels out the net effect.

To address the issue of residual fluid from one sub-circuit coming into the other—the fluid coming out of the dialyzer comes from the patient only, and therefore, it is perfectly safe to put that fluid back into the patient along with the sterile replacement fluid.

### Ultrafiltrate Pump Accuracy Required

As mentioned previously, during dialysis, additional fluid may be removed from the patient if required, in the form of ultrafiltrate (UF), and a UF pump is provided for this purpose in the system of present invention. Further, volumetric accuracy is maintained irrespective of how much or how little UF is removed.

When pumping out ultrafiltrate to remove excess fluid from the patient, if the system has a lower pump rate, such as of the order of 10 ml/min, as opposed to a high rate such as 200 ml/min, achieving a defined overall volumetric accuracy is easier. For example if the accuracy required is ±30 ml, then over a time period of 60 minutes, 600 ml will be pumped with a pump rate of 10 ml/min. This implies that the percentage accuracy achieved is 30 ml/600 ml=0.05 or 5%, which is reasonable to obtain. One of ordinary skill in the art would, however, appreciate that the system of present invention is capable of achieving the desired volumetric accuracy, regardless of the pump rate of the UF pump in the dialysis device.

While there has been illustrated and described what is at present considered to be a preferred embodiment of the present invention, it will be understood by those skilled in the art that various changes and modifications may be made, and equivalents may be substituted for elements thereof without departing from the true scope of the invention. In addition, many modifications may be made to adapt a particular situation or material to the teachings of the invention without departing from the central scope thereof. Therefore, it is intended that this invention not be limited to the particular embodiment disclosed as the best mode contemplated for carrying out the invention, but that the invention will include all embodiments falling within the scope of the appended claims.

We claim:

1. A system for maintaining volumetric balance of the fluid infused into a patient and the fluid removed from said patient during renal dialysis, the system comprising:
a first fluid circuit for infusing fluid into the patient;
a second fluid circuit for removing fluid from the patient;
a first pump configured to alternately operate on said first circuit and said second circuit;
a second pump configured to alternately operate on said second circuit and said first circuit; and
a controller for causing said first pump to alternatively operate on said first circuit and said second circuit and for causing said second pump to alternatively operate on said first circuit and said second circuit, wherein each of the said first pump and second pump operate only one circuit at a given time.

2. The system of claim 1, wherein the first pump delivers a higher amount of fluid per unit time than the second pump.

3. The system of claim 1, wherein said first and second pumps alternately operate on said first and second circuits for a time interval ‘T’, wherein ‘T’ is derived from an allowable difference in the amount of fluid delivered per unit time by the said first and second pumps.

4. The system of claim 1, wherein said first and second pumps are peristaltic pumps.

5. The system of claim 1, wherein the first and second pumps and first and second fluid circuits are implemented in the form of a disposable device.

6. The system of claim 1 further comprising a restrictor for equalizing a pressure differential between said first and second circuits.

7. The system of claim 6 wherein said restrictor is active and equalizes said pressure differential based upon a measured pressure differential derived from a first pressure sensor in said first circuit and from a second pressure sensor in said second first circuit.

8. The system of claim 1, wherein the system is a hemofiltration system.

9. The system of claim 1, wherein the system is a closed-loop, sorbent-based system.

10. A method for maintaining volumetric balance of the fluid infused into a patient and the fluid removed from said patient during renal dialysis, the method comprising:
using a first fluid circuit for infusing fluid into the patient;
using a second fluid circuit for removing fluid from the patient;
providing a first pump having an ability to alternately operate on said first circuit and said second circuit;
providing a second pump having an ability to alternately operate on said second circuit and said first circuit;
causing said first pump to alternatively operate on said first circuit and said second circuit; and
causing said second pump to alternatively operate on said first circuit and said second circuit, wherein each of the said first pump and second pump operate only one circuit at a given time.

11. The method of claim 10, wherein said first pump delivers a higher amount of fluid per unit time than the second pump.

12. The method of claim 10, which includes alternately operating said first and second pumps on said first and second circuits for a time interval ‘T’, wherein ‘T’ is derived from an allowable difference in the amount of fluid delivered per unit time by the said first and second pumps.

13. The method of claim 10, wherein the method is implemented by a disposable device.

14. The method of claim 10 further comprising the step of equalizing a pressure differential between said first and second circuits.

15. The method of claim 10 wherein said pressure differential is determined based upon a measured pressure differential derived from a first pressure sensor in said first circuit and from a second pressure sensor in said second first circuit.

16. A method for maintaining volumetric balance of the fluid infused into a patient and the fluid removed from said patient during renal dialysis, the method comprising:
using a first fluid circuit for infusing fluid into the patient;
using a second fluid circuit for removing fluid from the patient;
providing a first pump having an ability to alternately operate on said first circuit and said second circuit;
providing a second pump having an ability to alternately operate on said second circuit and said first circuit;
causing said first pump to alternatively operate on said first circuit and said second circuit;
causing said second pump to alternatively operate on said first circuit and said second circuit, wherein each of the said first pump and second pump operate only one circuit at a given time; and
equalizing a pressure differential between said first and second circuits, wherein said pressure differential is determined based upon a measured pressure differential derived from a first pressure sensor in said first circuit and from a second pressure sensor in said second first circuit.

17. The method of claim 16, wherein said first pump delivers a higher amount of fluid per unit time than the second pump.

18. The method of claim 16, which includes alternately operating said first and second pumps on said first and second circuits for a time interval ‘T’, wherein ‘T’ is derived from an allowable difference in the amount of fluid delivered per unit time by the said first and second pumps.

19. The method of claim 16, wherein said first and second pumps are peristaltic pumps.

20. The method of claim 16, wherein the method is implemented by a disposable device.
